(19) United States
(12) Patent Application Publication
(10) Pub. No.: US 2013/0183366 A1
(43) Pub. Date: Jul. 18, 2013

(54) SHEET PREPARATION FOR TISSUE ADHESION

(71) Applicants: Hideki Saga, Kumamoto-shi (JP); Tsutomu Hamuro, Kumamoto-shi (JP)

(72) Inventors: Hideki Saga, Kumamoto-shi (JP); Tsutomu Hamuro, Kumamoto-shi (JP)

(73) Assignee: THE CHEMO-SERO-THERAPEUTIC RESEARCH INSTITUTE, Kumamoto-shi (JP)

(21) Appl. No.: 13/811,096

(22) PCT Filed: Jul. 14, 2011
(86) PCT No.: PCT/JP2011/066099

§ 371 Date: Apr. 4, 2013

(30) Foreign Application Priority Data
Jul. 20, 2010 (JP) 2010-162979

(51) Int. Cl. A61F 13/00

(52) U.S. Cl. CPC A61F 13/00063
USPC 424/444; 427/2.31; 602/50; 602/45; 424/447; 424/94.64

(57) ABSTRACT

A sheet preparation characterized by that fibrinogen is held on one surface of a sheet-shaped support and thrombin is held on the other surface of the sheet-shaped support where fibrinogen is not held and that fibrinogen and thrombin are held separately from each other, and a process for preparing the same.

### TECHNICAL FIELD

The present invention relates to a sheet preparation consisting of a sheet-shaped support which comprises fibrinogen and thrombin as an active ingredient and a process for preparing the same. More specifically, the present invention relates to a sheet preparation used for tissue adhesion characterized by that fibrinogen is held on one surface of a sheet-shaped support and thrombin is held on the other surface of the sheet-shaped support, and a process for preparing the same.

### BACKGROUND ART

Fibrinogen is a very important coagulation factor which acts in the final stage of the blood coagulation cascade. Fibrinogen, e.g. upon activation of the coagulation system after an injury, is converted by thrombin from its soluble form into insoluble fibrin which plays an important role in hemostasis and wound healing.

Fibrinogen has importance in hemostasis and wound healing. For instance, fibrinogen has been used clinically as an intravenous dosage form in a replacement therapy against congenital and acquired fibrinogen deficiencies etc. to hamper a serious bleeding by increasing the fibrinogen level in blood. Additionally, in recent years, a fibrin adhesive consisting of fibrinogen and thrombin is used in a surgery as an adhesive for substitute of suture of soft organs such as the liver and the spleen or as an auxiliary agent for the suture. Fibrinogen has also widely been applied in other clinical set-up.

Such a fibrin adhesive, capable of adhering to a wound or a tissue surface, may enhance a tension strength of an adhesion site or a joined wound, may fully be absorbed within the living body and may promote the healing of wound.

A fibrin adhesive is not stable in a form of a solution and thus is used in clinical practice in a dosage form of a frozen solution or a lyophilized powder. Therefore, a commercially available preparation has to be thawed or rehydrated before application, in either of which a lot of time is wasted. Specifically, a fibrin adhesive that is prepared from a lyophilized product consists of a lyophilized powder of fibrinogen, a fibrinogen solution, a lyophilized powder of thrombin and a thrombin solution. When it takes time for dissolving the lyophilized powder and for preparing for applying and mixing the fibrinogen solution and the thrombin solution, there are concern that it might adversely affect a patient, especially in case of a surgical operation at emergency.

Additionally, to obtain a sufficient adhesive action as a fibrin adhesive, it is necessary to dissolve fibrinogen in a higher concentration. To be coagulated fully, the higher concentration of fibrinogen is the more favorable. However, there is a problem that such a high-concentration fibrinogen solution is not suitable for use in a surgical operation at emergency since it would take a long time to make the solution from a lyophilized fibrinogen preparation.

A sheet preparation consisting of a bioabsorbable sheet holding thrombin, and a fibrinogen solution is reported (Patent reference 1, Patent reference 2). The sheet preparation is used in clinical set-up of a surgical operation such that a sheet holding thrombin is immersed in a fibrinogen solution and then applied to the operation site. The sheet preparation may exert excellent hemostatic and adhesive effects. However, although thrombin is held on a sheet in advance, the sheet preparation may still not be used for emergent use since it would take time for preparing a fibrinogen solution.

Thus, a pharmaceutical preparation being looked for in clinical set-up is (1) a pharmaceutical preparation having high adhesive and hemostatic effects; (2) a dosage form that does not require time for preparation, i.e. an integrated sheet preparation in which fibrinogen and thrombin are held together. An integrated sheet preparation currently approved for clinical use includes a sheet preparation (TachoComb (registered trademark): CSL Behring) in which fibrinogen and thrombin are mixed and held on a support made of equine collagen. However, efficacy of said sheet preparation is limited and does not satisfy surgeons. Furthermore, a process for preparing said sheet preparation is extremely complicated (Patent reference 3). Thus, in clinical set-up, a pharmaceutical preparation is desired that can cope with surgical operation at emergency and has potent tissue-adhesive, sealing and hemostatic effects. Besides, inventing a more convenient process for preparing a sheet preparation would allow for provision of an inexpensive pharmaceutical preparation with potent tissue-adhesive, sealing and hemostatic effects in clinical set-up.

A sheet-shaped fibrin adhesive consisting of two-layered bioabsorbable supports, one layer holding fibrinogen as an effective ingredient and the other layer holding thrombin as an effective ingredient, is known (Patent reference 4). However, since said invention is consisted of two-layered supports, its handling for applying to the affected parts is troublesome and, in case of a microscopic operation, its application to the affected parts would be difficult. Besides, in case of emergent operation or irregular hemorrhage, it is also difficult to apply.

A carrier having at least one of physical properties of ventricles, i.e. an elastic module in a range of 5-100 N/cm, a density of 1-10 mg/cm³, a diameter of more than 0.75 mm and less than 4 mm, and/or a mean diameter of less than 3 mm, and a solid composition comprising said carrier that holds solid fibrinogen and solid thrombin are known (Patent reference 5). However, since said invention is not a sheet preparation, it is difficult to use the composition with pressure. In addition, said composition has a low tensile strength and a poor tissue sealing capacity. Besides, its handling is troublesome since an applicator is necessary for applying to the affected parts. Furthermore, as a consequence of holding of solid fibrinogen and solid thrombin on a carrier, the two ingredients may instantaneously react with each other to form fibrin so that insufficient adhesion to the tissue to be applied would result. For exerting a high adhesive effect, it is critical that fibrinogen, while penetrating into a tissue adhesion site, is reacted with thrombin to gelation.
Patent reference 1: WO 2004/064878
Patent reference 2: WO 2005/113030
Patent reference 3: Japanese Patent Publication No. 2004-520124
Patent reference 4: Japanese Patent Publication No. 2010-69031
Patent reference 5: Japanese Patent Publication No. 2007-190399

### DISCLOSURE OF THE INVENTION

### Technical Problem to be Solved by the Invention

Under these circumstances, an object of the present invention is to provide (1) a pharmaceutical preparation having high adhesive and hemostatic effects; (2) a dosage form that does not require time for preparation; (3) a pharmaceutical preparation that can easily be prepared and is inexpensive, i.e. an integrated sheet preparation in which fibrinogen and thrombin are held together, and a process for preparing the same. Specifically, an object of the present invention is to provide a sheet preparation that may exert more potent tissue-adhesive, sealing and hemostatic effects than the currently approved pharmaceutical preparations and is inexpensive, and a process for preparing the same.

### Means for Solving the Problems

Thus, viewing the above problems, the present inventors have earnestly studied and as a result found that an integrated sheet preparation in which fibrinogen and thrombin are held together possesses the above properties, which integrated sheet preparation is prepared by soaking one surface of a sheet-shaped support with a fibrinogen solution and lyophilizing the sheet-shaped support thereby let a lyophilized powder of fibrinogen be held on the sheet-shaped support, and then applying a powder dispersion of a thrombin powder to the other surface of the sheet-shaped support and drying the sheet-shaped support to thereby let a dry powder of thrombin be held on the sheet-shaped support. Said sheet preparation may hold fibrinogen at a high concentration per area of the surface that holds fibrinogen through holding a lyophilized dry powder of fibrinogen on the surface. Thus, said sheet preparation may exert more potent tissue-adhesive, sealing and hemostatic effects than the currently available pharmaceutical preparations. The present invention is explained in more detail hereinbelow.

The present invention included the following inventions.

[1] A sheet preparation characterized by that fibrinogen is held on one surface of a sheet-shaped support and thrombin is held on the other surface of the sheet-shaped support where fibrinogen is not held and that fibrinogen and thrombin are held separately from each other.

[2] The sheet preparation according to the above [1] wherein said sheet-shaped support is one having a thickness of at least 0.3 mm.

[3] The sheet preparation according to the above [1] or [2] wherein said sheet-shaped support is one made from a bioabsorbable material selected from the group consisting of polyglycolic acid, polylactic acid, polycaprolactone, polyglycerol-sebacic acid, polyhydroxy-alkanoic acid, polybutylene succinate, and a copolymer and a derivative thereof.

[4] The sheet preparation according to the above [3] wherein said sheet-shaped support is one made from polyglycolic acid.

[5] The sheet preparation according to any of the above [1] to [4] wherein said sheet-shaped support is one made from a non-woven fabric prepared by needle-punching a knit or a textile of a bioabsorbable material.

[6] The sheet preparation according to any of the above [1] to [5] wherein fibrinogen is a lyophilized powder of fibrinogen.

[7] The sheet preparation according to the above [6] wherein said lyophilized powder of fibrinogen is one prepared by applying or soaking a fibrinogen solution to one surface of the sheet-shaped support and then lyophilizing the sheet-shaped support.

[8] The sheet preparation according to any of the above [1] to [7] wherein said fibrinogen is held on the surface of the sheet-shaped support at 1.5 mg/cm² or more per area of the surface that holds fibrinogen.

[9] The sheet preparation according to any of the above [1] to [8] wherein said fibrinogen solution is one that contains 1% to 15% (w/v) fibrinogen.

[10] The sheet preparation according to any of the above [1] to [9] wherein thrombin is a thrombin powder.

[11] The sheet preparation according to the above [10] wherein said thrombin powder is one prepared by applying a powder dispersion in which a thrombin powder is dispersed in a solvent to the other surface of the sheet-shaped support where fibrinogen is not held and drying the sheet-shaped support.

[12] The sheet preparation according to the above [11] wherein said solvent is alcohols.

[13] The sheet preparation according to the above [12] wherein said alcohols are selected from the group consisting of ethanol, methanol, isopropyl alcohol, 1-butanol, 2-butanol, isobutyl alcohol, and isopentyl alcohol.

[14] The sheet preparation according to the above [13] wherein said alcohols are ethanol.

[15] The sheet preparation according to any of the above [1] to [14] wherein said thrombin is held on the surface of the sheet-shaped support at 0.28 U/cm² or more per area of the surface that holds thrombin.

[16] The sheet preparation according to any of the above [1] to [15] wherein said sheet preparation comprises at least one selected from the group consisting of Factor XIII, aprotinin, albumin, calcium chloride, a nonionic surfactant, and various amino acids.

[17] A process for preparing the sheet preparation as set forth in any of the above [1] to [16], said process comprising holding fibrinogen on one surface of a sheet-shaped support and holding thrombin on the other surface of the sheet-shaped support where fibrinogen is not held.

[18] A process for preparing a sheet preparation which comprises the steps:
(A) applying or soaking one surface of a sheet-shaped support with a fibrinogen solution and lyophilizing the sheet-shaped support to thereby let a lyophilized powder of fibrinogen be held on the sheet-shaped support, and
(B) applying a powder dispersion of a thrombin powder to the other surface of the sheet-shaped support and drying the sheet-shaped support to thereby let a thrombin powder be held on the sheet-shaped support; wherein fibrinogen and thrombin are separately held on the distinct surfaces of the sheet-shaped support.

[19] The process for preparing a sheet preparation according to the above [18] wherein said fibrinogen solution is applied in such an amount that said fibrinogen solution does not reach the other surface of the sheet-shaped support where a thrombin powder is held.

[20] A method for hemostasis which comprises using the sheet preparation as set forth in any of the above [1] to [16].

### EFFECTS OF THE INVENTION

In accordance with the present invention, a sheet preparation with a lyophilized fibrinogen which holds a thrombin powder and a process for preparing the same are provided. The sheet preparation according to the present invention may exert excellent effects as follows:
(1) It may exert more potent tissue-adhesive, sealing and hemostatic effects than the currently available pharmaceutical preparations;
(2) It may form a γ-dimer, which the currently available pharmaceutical preparations cannot form, to thereby enhance physical strength and stability of fibrin clot;
(3) It is a very convenient pharmaceutical preparation that does not require time for preparation and may be used in case of emergency;
(4) It is excellent in rehydration;
(5) It is bioabsorbable;
(6) It is excellent in flexibility and elasticity;
(7) It is excellent in holding the effective ingredients; and
(8) It can easily be prepared and is inexpensive.

### BRIEF DESCRIPTION OF DRAWINGS

FIG. 1 shows results of a test for evaluating an adhesive effect of the sheet preparation of the present invention.

FIG. 2 shows results of a test for evaluating an adhesive effect of the sheet preparation of the present invention.

FIG. 3 shows results of a test for evaluating an adhesive effect of the sheet preparation of the present invention.

FIG. 4 shows results of SDS-PAGE of the sheet preparation of the present invention.

FIG. 5 shows results of a test for evaluating a hemostatic effect of the sheet preparation of the present invention.

### BEST MODE FOR CARRYING OUT THE INVENTION

The sheet preparation of the present invention is one characterized by that fibrinogen is held on one surface of a sheet-shaped support and thrombin is held on the other surface of the sheet-shaped support where fibrinogen is not held and that fibrinogen and thrombin are held separately from each other.

Fibrinogen may be a dried powder, a lyophilized powder, or a gel containing fibrinogen, preferably a lyophilized powder. In addition to fibrinogen, fibrinogen may further include Factor XIII, aprotinin, albumin, sodium chloride, sodium citrate, a nonionic surfactant such as Polysorbate 80, various amino acids, glycerol, and the like.

In a process for holding fibrinogen, a fibrinogen solution may be applied or soaked to one of the surfaces of a sheet-shaped support which is then lyophilized so as to hold a lyophilized powder of fibrinogen or alternatively a lyophilized powder of fibrinogen may directly be applied to one of the surfaces of a sheet-shaped support. Preferably, a fibrinogen solution may be soaked to a sheet-shaped support which is then lyophilized. Under these circumstances, fibrinogen should not be applied, infiltrated or presented to the other surface of a sheet-shaped support.

Fibrinogen may preferably be held on the surface of a sheet-shaped support at 1.5 mg/cm² or more per area of the surface that holds fibrinogen, more preferably at 1.5 mg/cm² to 30 mg/cm², even more preferably at 1.5 mg/cm² to 15 mg/cm², even more preferably at 1.5 mg /cm² to 12 mg/cm², even more preferably at 3 mg/cm² to 12 mg/cm², and even more preferably at 6 mg/cm² to 12 mg/cm². An amount of fibrinogen to be held on a sheet-shaped support may suitably be varied depending on a thickness of a sheet-shaped support or a concentration of a fibrinogen solution.

A fibrinogen solution may preferably contain fibrinogen at 1% to 15% (w/v), more preferably at 1% to 12% (w/v), even more preferably at 2% to 12% (w/v), even more preferably at 3% to 12% (w/v), and even more preferably at 3% to 6% (w/v). A concentration of fibrinogen may suitably be varied depending on a thickness of a sheet-shaped support or an amount of fibrinogen to be held on a sheet-shaped support.

Thrombin may be a dried powder, a lyophilized powder, or a gel containing thrombin, preferably a dried powder. In addition to thrombin, thrombin may further include albumin, sodium chloride, sodium citrate, a nonionic surfactant such as Polysorbate 80, various amino acids, glycerol, and the like.

In a process for holding thrombin, a powder dispersion of a thrombin powder may be applied to a sheet-shaped support which is then dried or alternatively a thrombin powder may directly be applied to a sheet-shaped support. Preferably, a powder dispersion of a thrombin powder may be applied to a sheet-shaped support which is then dried to hold thrombin.

A thrombin powder may be applied to a sheet-shaped support at 0.28 U/cm² or more per area of the surface that holds thrombin, more preferably at 0.28 to 60 U/cm², even more preferably at 0.28 to 30 U/cm², even more preferably at 0.28 to 15 U/cm², even more preferably at 0.28 to 13.8 U/cm², even more preferably at 1.38 to 13.8 U/cm², and even more preferably at 4.6 to 13.8 U/cm². An amount of thrombin to be held on a sheet-shaped support may suitably be varied depending on an amount of fibrinogen to be held on a sheet-shaped support.

For a powder dispersion of a thrombin powder, an organic solvent may preferably be used as a main solvent. Preferably, an alcohol solvent such as ethanol, methanol, isopropyl alcohol, 1-butanol, 2-butanol, isobutyl alcohol, or isopentyl alcohol, or a solvent with a low boiling point that does not affect the human body may be used as a main solvent. In view of safety to the human body, ethanol may preferably be used as a main solvent.

A powder dispersion of a thrombin powder may preferably contain 5.6 U/mL to 1380 U/mL of thrombin, and 0.064 mg/mL to 64 mg/mL of calcium chloride. More preferably, it contains 27.6 U/mL to 276 U/mL of thrombin, and 0.64 mg/mL to 6.4 mg/mL of calcium chloride. A concentration of a thrombin powder may suitably be varied depending on a thickness of a sheet-shaped support or an amount of thrombin to be held on a sheet-shaped support.

For an order of fibrinogen and thrombin to be held on a sheet-shaped support, fibrinogen may firstly be held on a sheet-shaped support and thereafter thrombin may be held or alternatively thrombin may firstly be held on a sheet-shaped support and thereafter fibrinogen may be held, provided that fibrinogen and thrombin do not contact to, and react with, each other before application. In view of convenience and easiness in production, however, fibrinogen may preferably firstly be held on a sheet-shaped support and thereafter thrombin may be held. Specifically, one of the surfaces of a sheet-shaped support may be immersed in a fibrinogen solution to soak the fibrinogen solution and then the sheet-shaped support may be lyophilized to prepare a sheet holding fibrinogen. While preparation, it is necessary to adjust an amount of a fibrinogen solution to one that does not reach the other surface of a sheet-shaped support where a thrombin powder is held, taking into consideration an area and a thickness of a sheet-shaped support to be used. Thereafter, a powder dispersion of a thrombin powder dispersed in ethanol may be applied to the surface of a sheet-shaped support opposite to the surface where fibrinogen is held and the sheet-shaped support may be dried.

A thickness a sheet-shaped support may preferably be at least 0.15 mm, more preferably at least 0.3 mm, even more preferably at least 0.4 mm, even more preferably at least 0.8 mm, and even more preferably at least 1 mm. An upper limit of a thickness of a sheet-shaped support may suitably be varied depending on the tissue to which the sheet preparation is applied and an amount of fibrinogen to be applied.

A bioabsorbable material used for a sheet-shaped support may preferably be aliphatic polyesters. Specifically, a bioabsorbable material includes polylactic acid, polyglycolic acid, polycaprolactone, polyglycerol-sebacic acid, polyhydroxy-alkanoic acid, polybutylene succinate, and a copolymer and a derivative thereof. Preferably, a bioabsorbable material may be at least one selected from the group consisting of polyglycolic acid, polylactic acid, polycaprolactone, a copolymer and a derivative thereof. More preferable is polyglycolic acid and a derivative thereof.

A structure of a sheet-shaped support may be in the form of nonwoven fabric, sponge, textile, knit, braid or a composite thereof, among which nonwoven fabric is preferable.

Nonwoven fabric may preferably be one manufactured by, for instance, meltblown, needlepunching, spunbonding, flash spinning, and the like. Especially, preferable is nonwoven fabric manufactured by needle-punching piled woven or knitted fabric using bioabsorbable material (e.g. polyglycolic acid) into nonwoven fabric (Japanese Patent Publication No. 18579/1993).

A method for preparing fibrinogen, thrombin, albumin, aprotinin and Factor XIII as used herein is not particularly limited and includes e.g. separation from human blood or by genetic recombination technique.

The present invention is explained in more detail by means of the following Examples but is not limited thereto.

### EXAMPLE

### Example 1

### Process for Preparing Sheet Preparation

For a sheet-shaped support, a bioabsorbable sheet was used. Specifically, for a bioabsorbable sheet, Neoveil 015G (registered trademark) (Gunze; composition: polyglycolic acid; thickness 0.15 mm), Neoveil 03G (registered trademark) (Gunze; composition: polyglycolic acid; thickness 0.3 mm), and Neoveil 05G (registered trademark) (Gunze; composition: polyglycolic acid; thickness 0.5 mm) were used.

A fibrinogen solution I was prepared to contain about 12.0% (w/v) fibrinogen, about 90 IU/mL Factor XIII, 1.5% (w/v) human serum albumin, 2.6% (w/v) sodium chloride, 1.8% (w/v) trisodium citrate, 0.5% (w/v) isoleucine, 0.7% (w/v) glycine, 1.6% (w/v) arginine hydrochloride, 1.2% (W/V) sodium glutamate, 0.03% (w/v) Polysorbate 80, 1.0% glycerol at pH 6.7 to 6.9. A fibrinogen solution II is one that is obtained by diluting the fibrinogen solution I by two-fold with water for injection to give a solution of about 6.0% (w/v) fibrinogen, to which glycerol is added to make a solution of 1.0% glycerol. A fibrinogen solution III is one that is obtained by diluting the fibrinogen solution I by four-fold with water for injection to give a solution of about 3.0% (w/v) fibrinogen, to which glycerol is added to make a solution of 1.0% glycerol. For a lyophilized powder of thrombin, one from Bolheal (registered trademark) (THE CHEMO-SERO-THERAPEUTIC RESEARCH INSTITUTE) was used. For calcium chloride powder, calcium chloride dihydrate (nacalai tesque) was used. For a solution of powder dispersion, ethanol (Wako Pure Chemical Industries, Ltd.; purity: 99.5%) was used.

Bioabsorbable sheets of a size 2.5×2.0 cm were placed on Petri dishes. For a sheet-shaped support of a thickness 0.8 mm, Neoveil 03G and Neoveil 05G were piled up. For a sheet-shaped support of a thickness 0.45 mm, Neoveil 015G and Neoveil 03G or three Neoveil 015G were piled up. For a sheet-shaped support of a thickness 0.3 mm, Neoveil 03G was used or two Neoveil 015G were piled up.

For Inventions A, B, J, K and L, bioabsorbable sheets were soaked with a fibrinogen solution I at 50 μL/cm² of fibrinogen per area of the surface that holds fibrinogen and were lyophilized to prepare fibrinogen-holding sheets. For Inventions C and D, bioabsorbable sheets were soaked with a fibrinogen solution I at 100 μL/cm² of fibrinogen per area of the surface that holds fibrinogen and were lyophilized to prepare fibrinogen-holding sheets. For Invention E, a bioabsorbable sheet was soaked with a fibrinogen solution III at 50 μL/cm² of fibrinogen per area of the surface that holds fibrinogen and was lyophilized to prepare a fibrinogen-holding sheet. For Invention F, a bioabsorbable sheet was soaked with a fibrinogen solution III at 100 μL/cm² of fibrinogen per area of the surface that holds fibrinogen and was lyophilized to prepare a fibrinogen-holding sheet. For Invention G, a bioabsorbable sheet was soaked with a fibrinogen solution II at 50 μL/cm² of fibrinogen per area of the surface that holds fibrinogen and was lyophilized to prepare a fibrinogen-holding sheet.

The surface of the respective fibrinogen-holding sheets opposite to the surface that holds fibrinogen was applied with a powder solution of a lyophilized powder of thrombin and calcium chloride powder dispersed in ethanol and the sheets were dried. An amount of a lyophilized powder of thrombin to be applied per area of the surface that holds thrombin was as follows: 13.8 U/cm² for Inventions A, B, C, D, E, F and G; 0.28 U/cm² for Invention J; 1.38 U/cm² for Invention K; and 4.6 U/cm² for Invention L. An amount of calcium chloride powder to be applied was 0.32 mg/cm².

The conditions of the respective Inventions are summarized in the table below.

TABLE 1

| *1 | *2 | *3 | *4 | *5 | *6 |
|---|---|---|---|---|---|
| A | 0.3 | I (12%) | 50 | 6 | 13.8 |
| B | 0.45 | I (12%) | 50 | 6 | 13.8 |
| C | 0.45 | I (12%) | 100 | 12 | 13.8 |
| D | 0.8 | I (12%) | 100 | 12 | 13.8 |
| E | 0.3 | III (3%) | 50 | 1.5 | 13.8 |
| F | 0.3 | III (3%) | 100 | 3 | 13.8 |
| G | 0.3 | II (6%) | 50 | 3 | 13.8 |
| J | 0.3 | I (12%) | 50 | 6 | 0.28 |
| K | 0.3 | I (12%) | 50 | 6 | 1.38 |
| L | 0.3 | I (12%) | 50 | 6 | 4.6 |

*1: Invention
*2: A thickness of sheet-shaped support (mm)
*3: Fibrinogen solution (fibrinogen conc. w/v)
*4: An amount of fibrinogen solution (μL/cm²)
*5: An amount of fibrinogen (mg/cm²)
*6: An amount of thrombin (U/cm²)

### Example 2

### Test for Evaluating Adhesive Force of Sheet Preparation

For saline, saline of Japanese Pharmacopoeia (Otsuka Pharmaceutical Co., Ltd.) was used. For water for injection, water for injection of Japanese Pharmacopoeia (The Chemo-Sero-Therapeutic Research Institute) was used. As a control of a sheet preparation, TachoComb (registered trademark) (CSL Behring) was used.

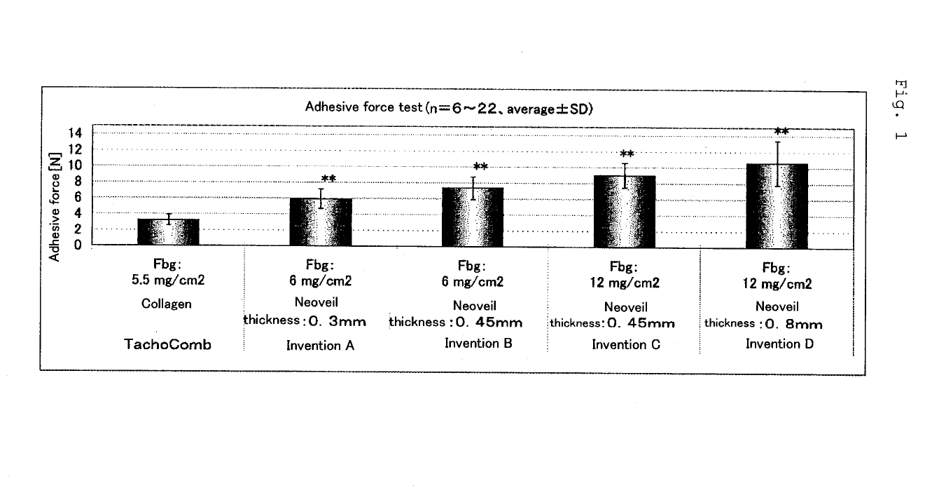

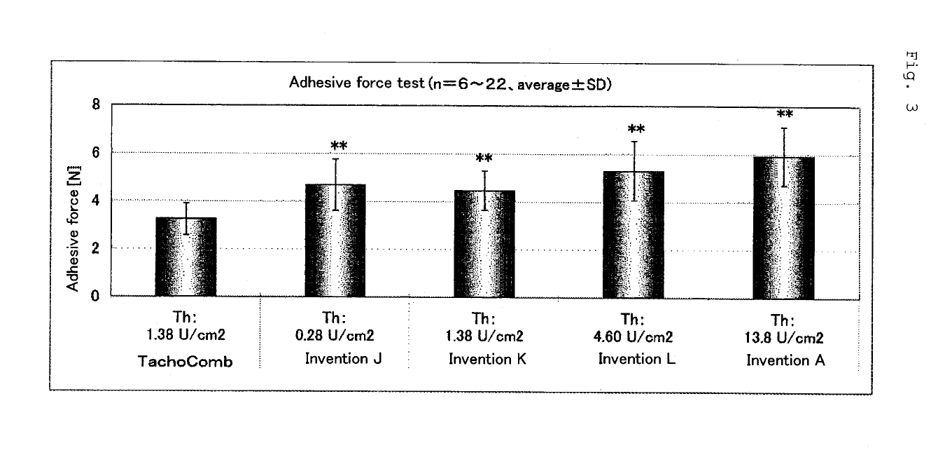

Two sheets of pig dermis were fixed by fixtures separately (adhesion area: 2.5×2.5 cm), 50 μL/cm² of saline was applied to the sheets, and a sheet preparation was placed thereon. After pressure for five minutes, one of the fixtures was immediately pulled horizontally, a maximum tensile strength (N: Newton) was determined and the obtained value was recorded as an adhesive force of the sheet preparation. N number was 22 for TachoComb, 5 for Inventions E/G, 6 for Inventions A/B/D/F/J/K/L, and 8 for Invention D. The results are shown in FIGS. 1, 2 and 3.

As is clear from FIGS. 1, 2 and 3, the adhesive force of Inventions showed significant difference from TachoComb by U-test of Mann-Whitney (**P<0.01). Therefore, it was proved that the sheet preparation according to the present invention exhibited more potent tissue-adhesive, sealing and hemostatic effects than those of the preparation currently clinically approved.

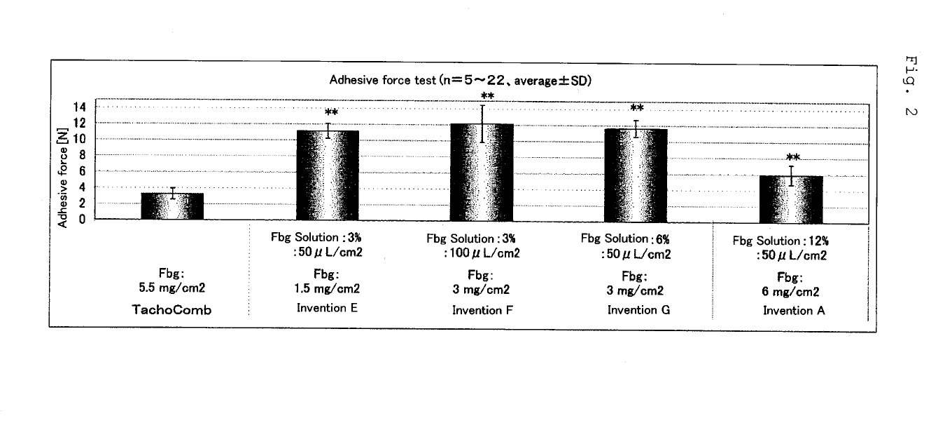

Besides, in FIG. 2, Inventions E, F and G exhibited more excellent adhesive effect than Invention A in spite of a smaller amount of fibrinogen applied to a sheet-shaped support for Inventions E, F and G. Although it can easily be conceived that the presence of a higher concentration of fibrinogen on a sheet-shaped support would provide a higher adhesive force, it was demonstrated that a factor other than an amount of fibrinogen on a sheet-shaped support (i.e. a concentration of a fibrinogen solution to be applied) would also contribute to an adhesive force, which was a surprising finding to the present inventors.

### Example 3

### SDS-PAGE of Sheet Preparation

[Preparation of Samples before Gelation: 0 minute]

Tested were TachoComb and Invention B prepared in Example 1. Sheets of 0.5×0.8 cm=0.4 cm² were prepared and placed in a sample tube. A solution for reduction was added to the sample tube. While properly stirring the solution containing the sheets, the sheets were dissolved at room temperature for 48 hours. After boiling, phoresis, staining (CBB) and discoloring were carried out.

[Preparation of Samples after Gelation: 5, 60 Minutes]

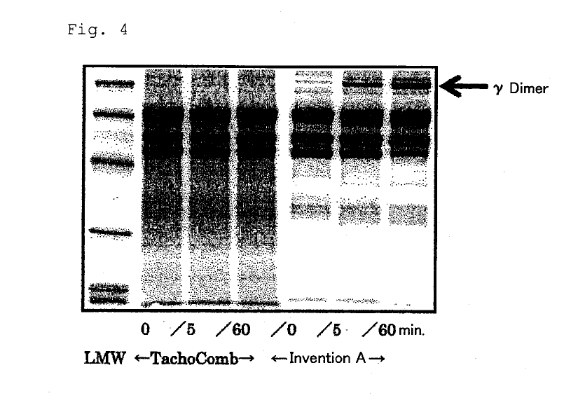

Tested were TachoComb and Invention A prepared in Example 1. Sheets of 0.5×0.8 cm=0.4 cm² were prepared and placed in a sample tube. Saline (40 μL) was added to the sample tube to dissolve and to be subject to gelation. Five minutes or sixty minutes after addition of saline and gelation, a solution for reduction was added to quench the gelation. While properly stirring the solution containing the gel sheets, the sheets were dissolved at room temperature for 48 hours. After boiling, phoresis, staining (CBB) and discoloring were carried out. The results are shown in FIG. 4.

As is clear from FIG. 4, Inventions formed a γ dimer which the conventional preparation could not form. Therefore, it was proved that the sheet preparation according to the present invention had enhanced physical strength and stability of fibrin clot due to formation of cross-linking within fibrin gel-matrix to thereby exhibit potent tissue-adhesive, sealing and hemostatic effects.

### Example 4

### Test for Evaluating Hemostatic Effect of Sheet Preparation

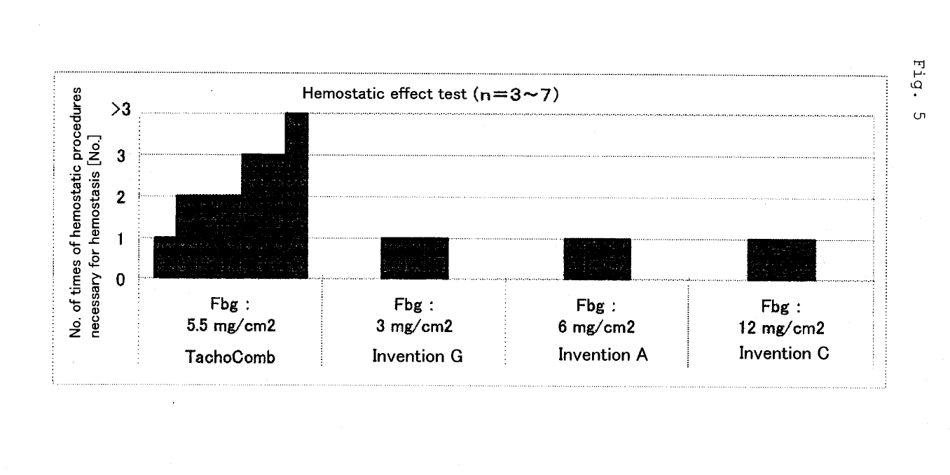

An animal model for evaluating hemostatic effect was prepared using SD male rat of 10-12 weeks old. Under anesthesia with xylazine hydrochloride and ketamine hydrochloride, the abdomen was incised along the median line. The abdominal aorta was exposed using gauze. Sodium heparin was administered via the abdominal vena cava at 300 U/kg. Two minutes after administration of heparin, the abdominal aorta was punctured with an injection needle to cause bleeding. Immediately thereafter, groups of test sheets of 1×1 cm were applied and pressed for 3 minutes. After pressure, the occurrence of rebleeding was observed for 3 minutes. When rebleeding was observed, the test sheet group was removed and a new test sheet group was applied and the hemostatic procedure was carried out. The hemostatic effect was evaluated with a number of times of the hemostatic procedures necessary for hemostasis. number was 7 for TachoComb, and 3 for Inventions A/C/G. The results are shown in FIG. 5.

As is clear from FIG. 5, inventions had a higher hemostatic effect than TachoComb. Therefore, it was proved that the sheet preparation according to the present invention exhibited more potent tissue-adhesive, sealing and hemostatic effects than those of the preparation currently clinically approved.

### INDUSTRIAL APPLICABILITY

The sheet preparation according to the present invention is a very convenient pharmaceutical preparation that does not require time for preparation and may be used in case of emergency for attaining tissue adhesion, sealing and hemostasis during surgical operation. It may exert more potent tissue-adhesive, sealing and hemostatic effects than the currently available pharmaceutical preparations and may form a γ-dimer, which the currently available pharmaceutical preparations cannot form, to thereby enhance physical strength and stability of fibrin clot. Besides, since it can more easily be prepared than the prior art, a sheet preparation that is inexpensive and highly effective may be provided. The present invention may provide a sheet preparation that is highly effective, convenient and inexpensive and a process for preparing the same.

1. A sheet preparation, comprising:
fibrinogen on a first surface of an integrated sheet-shaped support and
thrombin on a second surface of the integrated sheet-shaped support,
wherein the fibrinogen and the thrombin are separate from each other.

2. The sheet preparation of claim 1, wherein the integrated sheet-shaped support has a thickness of at least 0.3 mm.

3. The sheet preparation of claim 1,
wherein the integrated sheet-shaped support comprises a bioabsorbable material selected from the group consisting of polyglycolic acid, polylactic acid, polycaprolactone, polyglycerol-sebacic acid, polyhydroxy-alkanoic acid, polybutylene succinate, a polyglycolic acid copolymer, a polylactic acid copolymer, a polycaprolactone copolymer, a polyglycerol-sebacic acid copolymer, a polyhydroxy-alkanoic acid copolymer, a polybutylene succinate copolymer, a polyglycolic acid derivative, a polylactic acid derivative, a polycaprolactone derivative, a polyglycerol-sebacic acid derivative, a polyhydroxy-alkanoic acid derivative, and a polybutylene succinate derivative.

4. The sheet preparation of claim 3, wherein the integrated sheet-shaped support comprises polyglycolic acid.

5. The sheet preparation of claim 3, wherein the integrated sheet-shaped support comprises a non-woven fabric obtained by a process comprising needle-punching a knit or by needle-punching a textile of the bioabsorbable material.

6. The sheet preparation of claim 1, wherein the fibrinogen is a lyophilized fibrinogen powder.

7. The sheet preparation of claim 6, wherein the lyophilized fibrinogen powder is obtained by a process comprising:
applying or soaking a fibrinogen solution to the first surface of the integrated sheet-shaped support and
lyophilizing the integrated sheet-shaped support.

8. The sheet preparation of claim 1, wherein the fibrinogen on the first surface is present in an amount of at least 1.5 mg/cm².

9. The sheet preparation of claim 7, wherein the fibrinogen solution comprises from 1% to 15% (w/v).

10. The sheet preparation of claim 1, wherein the thrombin is a thrombin powder.

11. The sheet preparation of claim 10, wherein the thrombin powder is obtained by a process comprising:
applying a thrombin powder dispersion dispersed in a solvent to the second surface and
drying the integrated sheet-shaped support.

12. The sheet preparation of claim 11, wherein the solvent is an alcohol.

13. The sheet preparation of claim 12, wherein the alcohol is selected from the group consisting of ethanol, methanol, isopropyl alcohol, 1-butanol, 2-butanol, isobutyl alcohol, and isopentyl alcohol.

14. The sheet preparation of claim 13, wherein the alcohol is ethanol.

15. The sheet preparation of claim 1, wherein the thrombin on the second surface is present in an amount of at least 0.28 U/cm².

16. The sheet preparation of claim 1, further comprising, at least one selected from the group consisting of Factor XIII, aprotinin, albumin, calcium chloride, a nonionic surfactant, and an amino acid.

17. A process for preparing the sheet preparation claim 1, the process comprising:
holding fibrinogen on the first surface and
holding thrombin on the second surface.

18. A process for preparing a sheet preparation, comprising:
applying or soaking a first surface of a sheet-shaped support with a fibrinogen solution and lyophilizing the sheet-shaped support, thereby obtaining a lyophilized fibrinogen powder thereon, and
applying a thrombin powder dispersion to a second surface of the sheet-shaped support and drying the sheet-shaped support, thereby holding the thrombin powder thereon,
wherein fibrinogen and thrombin are separately on different surfaces of the sheet-shaped support.

19. The process of claim 18, wherein the fibrinogen solution does not reach the second surface.

20. (canceled)

21. A sheet preparation, comprising:
fibrinogen on a first surface of an integrated sheet-shaped support with a thickness of from 0.3 mm to 0.8 mm and
thrombin on a second surface of the integrated sheet-shaped support,
wherein the fibrinogen and the thrombin are separate from each other.

22. The sheet preparation of claim 21,
wherein the integrated sheet-shaped support comprises a bioabsorbable material selected from the group consisting of polyglycolic acid, polylactic acid, polycaprolactone, polyglycerol-sebacic acid, polyhydroxy-alkanoic acid, polybutylene succinate, a polyglycolic acid copolymer, a polylactic acid copolymer, a polycaprolactone copolymer, a polyglycerol-sebacic acid copolymer, a polyhydroxy-alkanoic acid copolymer, a polybutylene succinate copolymer, a polyglycolic acid derivative, a polylactic acid derivative, a polycaprolactone derivative, a polyglycerol-sebacic acid derivative, a polyhydroxy-alkanoic acid derivative, and a polybutylene succinate derivative.

23. The sheet preparation of claim 22, wherein the integrated sheet-shaped support comprises polyglycolic acid.

24. The sheet preparation of claim 22, wherein the integrated sheet-shaped support comprises a non-woven fabric obtained by a process comprising needle-punching a knit or by needle-punching a textile of the bioabsorbable material.

25. The sheet preparation of claim 21, wherein the fibrinogen is a lyophilized fibrinogen powder.

26. The sheet preparation of claim 25, wherein the lyophilized fibrinogen powder obtained by a process comprising:
applying or soaking a fibrinogen solution to the first surface of the integrated sheet-shaped support and
lyophilizing the integrated sheet-shaped support.

27. The sheet preparation of claim 21, wherein the fibrinogen on the first surface is present in an amount of at least 1.5 mg/cm².

28. The sheet preparation of claim 26, wherein the fibrinogen solution comprises from 1% to 15% (w/v).

29. The sheet preparation of claim 21, wherein the thrombin is a thrombin powder.

30. The sheet preparation of claim 29, wherein the thrombin powder is obtained by a process comprising:
applying a thrombin powder dispersion dispersed in a solvent to the second surface and
drying the integrated sheet-shaped support.

31. The sheet preparation of claim 30, wherein the solvent is an alcohol.

32. The sheet preparation of claim 31, wherein the alcohol is selected from the group consisting of ethanol, methanol, isopropyl alcohol, 1-butanol, 2-butanol, isobutyl alcohol, and isopentyl alcohol.

33. The sheet preparation of claim 32, wherein the alcohol are ethanol.

34. The sheet preparation of claims 21, wherein the thrombin on the second surface is present in an amount of at least 0.28 U/cm².

35. The sheet preparation of claim 21, further comprising, at least one selected from the group consisting of Factor XIII, aprotinin, albumin, calcium chloride, a nonionic surfactant, and an amino acid.

36. A sheet preparation, comprising:
fibrinogen for an amount of at 1.5 to 3.0 mg/cm² on a first surface of an integrated sheet-shaped support and
thrombin on a second surface of the integrated sheet-shaped support,
wherein that fibrinogen and the thrombin are separate from each other.

37. The sheet preparation of claim 36, wherein the integrated sheet-shaped support has a thickness of at least 0.3 mm.

38. The sheet preparation of claim 36,
wherein the integrated sheet-shaped support comprises a bioabsorbable material selected from the group consisting of polyglycolic acid, polylactic acid, polycaprolactone, polyglycerol-sebacic acid, polyhydroxy-alkanoic acid, polybutylene succinate, a polyglycolic acid copolymer, a polylactic acid copolymer, a polycaprolactone copolymer, a polyglycerol-sebacic acid copolymer, a polyhydroxy-alkanoic acid copolymer, a polybutylene succinate copolymer, a polyglycolic acid derivative, a polylactic acid derivative, a polycaprolactone derivative, a polyglycerol-sebacic acid derivative, a polyhydroxy-alkanoic acid derivative, and a polybutylene succinate derivative.

39. The sheet preparation of claim 38, wherein the integrated sheet-shaped support comprises polyglycolic acid.

40. The sheet preparation of claim 38, wherein the integrated sheet-shaped support comprises a non-woven fabric obtained by a process comprising needle-punching a knit or by needle-punching a textile of the bioabsorbable material.

41. The sheet preparation of claim 36, wherein the fibrinogen is a lyophilized fibrinogen powder.

42. The sheet preparation of claim 41, wherein the lyophilized fibrinogen powder is obtained by a process comprising:
applying or soaking a fibrinogen solution to the first surface of the integrated sheet-shaped support and
lyophilizing the integrated sheet-shaped support.

43. The sheet preparation of claim 41, wherein the fibrinogen solution comprises from 3% to 6% (w/v).

44. The sheet preparation of claim 36, wherein the thrombin is a thrombin powder.

45. The sheet preparation of claim 44, wherein the thrombin powder is obtained by a process comprising:
applying a thrombin powder dispersion dispersed in a solvent to the second surface and
drying the integrated sheet-shaped support.

46. The sheet preparation of claim45, wherein the solvent is an alcohol.

47. The sheet preparation of claim 46, wherein the alcohol is selected from the group consisting of ethanol, methanol, isopropyl alcohol, 1-butanol, 2-butanol, isobutyl alcohol, and isopentyl alcohol.

48. The sheet preparation of claim 47, wherein the alcohol is ethanol.

49. The sheet preparation of claim 36, wherein the thrombin on the second surface is present in an amount of at least 0.28 U/cm².

50. The sheet preparation of claim 36, further comprising, at least one selected from the group consisting of Factor XIII, aprotinin, albumin, calcium chloride, a nonionic surfactant, and an amino acid.
